(19) United States
(12) Patent Application Publication
(10) Pub. No.: US 2026/0212982 A1
(43) Pub. Date: Jul. 23, 2026

(54) INFORMATION PROCESSING DEVICE

(71) Applicant: Hitachi, Ltd., Chiyoda-ku, Tokyo (JP)

(72) Inventors: Takayuki NOZAKI, Tokyo (JP); Akio KANI, Tokyo (JP); Keisuke HIRAYAMA, Tokyo (JP)

(21) Appl. No.: 19/139,043

(22) PCT Filed: May 8, 2023
(86) PCT No.: PCT/JP2023/017233

§ 371 Date: Jun. 13, 2025

(51) Int. Cl. G16H 20/10

(52) U.S. Cl. CPC G16H 20/10

(57) ABSTRACT

A processing device includes an input device, an output device, a processor, and a storage device. The input device receives quality information including a plurality of parameters related to a plurality of cell-processed products as an input, the quality information includes a parameter related to at least one of production information, treatment information, treatment result information, and transport information related to the cell-processed products, the storage device stores a predetermined quality standard and the quality information, the processor calculates a first region based on the quality standard, calculates a quality state of the cell-processed products based on the first region and the quality information, and calculates a corresponding range of one or more types of other parameters based on a range designated by the one or more types of parameters, and the output device outputs the corresponding range.

### TECHNICAL FIELD

The present invention relates to an information processing device, and more particularly to an information processing device for processing parameters relating to a cell-processed product. For example, in the case of the cell-processed product, a relationship between parameters such as production data and quality is visualized to control the quality.

### BACKGROUND ART

Regenerative medicine is a medical treatment that uses regenerated tissues or cells to restore dysfunctional or damaged tissues that are difficult to treat using methods in the related art. The series of steps leading up to treatment involves collecting a biological sample from a patient himself or herself or from another person, for example, at a medical institution. After collection, the biological sample is transported to the cell processing facility (CPF). At the CPF, the biological sample is subjected to separation, purification, gene introduction, and the like, and cells are then grown and organized by culture and the like, and a cell-processed product that meets a quality evaluation standard is transported to the medical institutions and the like for use in patient treatment.

In the related art, the production of the cell-processed product in the regenerative medicine is based on the concept of quality by test (QbT), similar to a pharmaceutical product, with quality guaranteed by results of final quality testing after production. Recently, the pharmaceutical product has been moving towards introducing quality by design (QbD), which builds quality into a product development process. The QbD secures quality by understanding a product, its development process and production process, and by managing and developing the production process of the product. A quality target product profile (QTPP) of a product to be developed is clarified, a quality attribute (QA) required for the product with respect to the QTPP is extracted, and a critical quality attribute (CQA), which is a quality attribute that is particularly important for securing quality, is identified. A critical material attribute (CMA) and a critical process parameter (CPP) from a material attribute (MA) and a process parameter (PP) that affect the COA are identified to develop a management strategy. Further, a design space is established that allows production at any scale or lot. The design space is an interaction with a multi-dimensional combination of input variables (the COA and the like) of a production process and the like which are proven to secure quality. Validity of the management strategy is continuously verified and improved. The introduction of the QbD is also being examined for the regenerative medicine, but the progress is slow due to the fact that, compared to the pharmaceutical product, the regenerative medicine uses the cells and the biological samples as raw materials, making it difficult to standardize the quality, and at present many processes are operated manually, which can lead to a variation in an operation content.

In order to introduce the QbD into the regenerative medicine, it is necessary to accumulate and manage various kinds of information on a life cycle of a cell-processed product, such as each of processes of collection, purification, gene introduction, culture, concentration, and transport, transplantation, material management of raw materials and the like, and clinical information such as adverse events after transplantation and/or safety information, to understand a relationship with quality characteristics, variability characteristics, treatment outcomes, and the like, to find an index or the like that has an influence on quality, and to develop a management strategy. However, as described above, the regenerative medicine uses the cells and the biological samples as the raw materials, whose quality is difficult to standardize, and at present many of the processes are operated manually, and thus it is difficult to achieve understanding of causal relationships and mechanisms in the life cycle of the cell-processed product, and finding of the index having a strong influence on the quality characteristics, the variability characteristics, the treatment outcome, and the like, from various kinds of information on the life cycle. In addition, it is difficult to understand the relationship between parameters such as production data and quality.

PTL 1 discloses a method for accumulating information on production processes and the like based on QbD for a pharmaceutical product and determining whether quality is met. Further, PTL 2 discloses a method for improving production conditions based on accumulated production information, raw material information, and the like, for regenerative medicine. However, the information accumulated in both PTLs is mainly about the production process. It is not assumed that not only the entire processes related to the production but also material management of a raw material or the like and the accumulation of various kinds of information in a life cycle of a cell-processed product or the like, such as clinical information after transplantation, are performed. There is no mention of a method for finding factors that have a strong influence on quality characteristics, variability characteristics, treatment outcome, and the like, from various kinds of information in the life cycle in order to understand causal relationships and mechanisms in the life cycle. In the regenerative medicine, the production process is diverse and complicated, making it difficult to understand the relationships between each piece of information in the life cycle. For example, there is no mention of a method for managing quality by visualizing a relationship between parameters such as production data and quality.

### CITATION LIST

### Patent Literature

PTL 1: WO 2020/158581
PTL 2: WO 2013/008733

### SUMMARY OF INVENTION

### Technical Problem

As shown in PTLs 1 and 2, information on the production process and the like can be accumulated based on the QbD, and it is possible to determine whether the quality is met and to improve the production condition and the like. However, it is not assumed that various kinds of information on the life cycle of the cell-processed product, such as raw materials other than the production process and the clinical information after transplantation, will be accumulated as parameters.

In addition, it is not assumed that, in order to understand the causal relationships and mechanisms in the life cycle, it is possible to find parameters that have a strong influence on quality characteristics, variability characteristics, treatment outcomes, and the like from various kinds of information in the life cycle, or to recognize the relationships between each of parameters in the life cycle.

Further, it is not assumed that the relationship between each of the parameters will be visualized.

The invention has been made to solve such problems, and an object thereof is to provide an information processing device that can generate more useful information regarding a relationship between parameters of a cell-processed product.

### Solution to Problem

An example of an information processing device according to the present disclosure is an information processing including an input device; an output device; a processor; and a storage device. The input device receives quality information including a plurality of parameters related to a plurality of cell-processed products as an input, the quality information includes a parameter related to at least one of production information, treatment information, treatment result information, and transport information related to the cell-processed products, the storage device stores a predetermined quality standard and the quality information, the processor calculates a first region based on the quality standard, calculates a quality state of the cell-processed products based on the first region and the quality information, and calculates a corresponding range of one or more types of other parameters based on a range designated by the one or more types of parameters, and the output device outputs the corresponding range.

An example of a program according to the invention causes a computer to function as the above-described information processing device.

An example of an information processing method according to the invention includes: a step of receiving, information including by an input device, quality a plurality of parameters related to a plurality of cell-processed products as an input, the quality information including a parameter related to at least one of production information, treatment information, treatment result information, and transport information related to the cell-processed products; a step of storing, by a storage device, a predetermined quality standard and the quality information; a step of calculating, by a processor, a first region based on the quality standard; a step of calculating, by the processor, a quality state of the cell-processed product based on the first region and the quality information; a step of calculating, by the processor, a corresponding range of one or more types of other parameters based on a range designated by the one or more types of parameters; and a step of outputting, by an output device, the corresponding range.

### Advantageous Effects of Invention

An information processing device according to the invention can generate more useful information regarding a relationship between parameters of a cell-processed product.

For example, the information processing device according to the invention can visualize a relationship between each parameter related to production or the like as an input and quality as an output. By changing a range of each parameter serving as the input in various patterns, it is possible to visualize how other parameters of the input and the quality serving as the output move in a design space.

A distance between a center of gravity and/or a boundary of the design space and a point plotted with the quality of a certain parameter can be digitized as product stability, and a corresponding range of the product stability with respect to the corresponding range of each parameter can be digitized.

Based on these values, it is possible to rank priorities of items to be improved in production and the like.

A user of the information processing device can give priority to improving the parameters that have the greatest influence on quality. Examples of a content of improvement include environments such as production parameters, selection of a device to be used for production, educational training content to an operator, a layout change of a device in a cell production room, and cleanliness. As a result, it is possible to stabilize the quality of a cell-processed product or the like.

### BRIEF DESCRIPTION OF DRAWINGS

FIG. 1 is a configuration diagram of a quality management system according to Embodiment 1 of the invention.

FIG. 2A is a diagram showing an example of factors that generate information in a life cycle of a cell-processed product.

FIG. 2B is a diagram showing an example of quality information.

FIG. 3 is a diagram showing a distance between a center of gravity and a boundary of a design space.

FIG. 4 is an example of a three-dimensional design space and a one-dimensional design space.

FIG. 5A is a display example of statistical information of the quality information.

FIG. 5B is a display example showing a product position with respect to the design space.

FIG. 5C is a display example of the quality information on a designated cell-processed product.

FIG. 5D is a display example showing the product position of the designated cell-processed product with respect to the design space.

FIG. 5E is a display example of the quality information on a plurality of cell-processed products designated from a graph.

FIG. 5F is a display example of the quality information on a plurality of cell-processed products designated from a table.

FIG. 5G is a display example showing the product positions of the plurality of cell-processed products designated with respect to the design space.

FIG. 5H is a display example showing a distribution of the quality information when a parameter range of the cell-processed product is changed.

FIG. 5I is a display example showing a distribution of the quality information when a plurality of parameter ranges of the cell-processed product are changed.

FIG. 6 is a flowchart showing an operation of the quality management system according to Embodiment 1.

FIG. 7 is a flowchart showing an operation of a quality management system according to Embodiment 4.

### DESCRIPTION OF EMBODIMENTS

In order to achieve the above object, the invention has the following configuration. An object, a feature, an advantage, and an idea of the invention are apparent to those skilled in the art from the description of the specification, and those skilled in the art can easily reproduce the invention from the description of the specification. Specific embodiments and the like of the invention described below show preferred embodiments of the invention, and are shown for illustration and description, and the invention is not limited thereto. It is apparent to those skilled in the art that various modifications can be made based on the description of the specification within the intention and scope of the invention disclosed in the specification.

### Embodiment 1

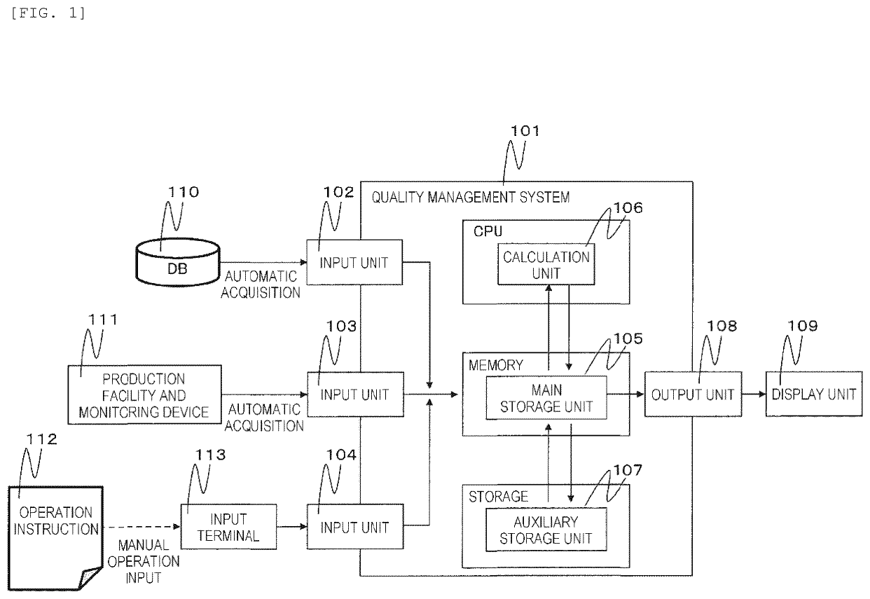

FIG. 1 shows a configuration of a quality management system 101 (information processing device) according to Embodiment 1. The quality management system 101 includes input units 102, 103, and 104 (input devices), an output unit 108 (output device), a calculation unit 106 (processor), a main storage unit 105 (storage device), and an auxiliary storage unit 107 (storage device).

The quality management system 101 handles information related to a cell-processed product. In the specification, the term “cell-processed product” widely includes products produced using cells or cell tissues, and particularly includes regenerative medicine products and the like as defined in the Pharmaceuticals and Medical Devices Act (PMD Act). The unit of the cell-processed product can be freely defined, but for example, one production lot can be defined as one unit of the cell-processed product.

The input units 102, 103, and 104 have a mechanism for taking in various types of data by linking with other systems and facility devices or by corresponding to manual input of data. Although each of parameters related to the production or the like is taken in as an input, three types of input units may be used according to a take-in method. The data taken therefrom is temporarily stored in the main storage unit 105 in the memory.

Further, the acquired data is processed and/or linked in the calculation unit 106 within a central processing unit (CPU), thereby giving value to the data.

For example, an influence on other parameters when each parameter as an input is changed (a situation of passively changing and responding) is calculated, and in particular, an influence of a change in quality as an output in the design space is calculated. A distance between the center of gravity of the design space and/or the boundary and a point plotted with the quality of a certain parameter is calculated as the product stability. A corresponding range or the like of the product stability with respect to a change state of each parameter is calculated. Based on these values, it is possible to rank priorities of items to be improved in production and the like.

A result and original data that is taken in are stored in the auxiliary storage unit 107 in the storage. The result and the data are output to the outside of the quality management system 101 via the output unit 108, and the data is displayed on the display unit 109. In one modification, the display unit 109 may be implemented as an output device of the quality management system 101. A relationship between each of the parameters related to the production taken in as an input and the quality obtained as an output is displayed using various calculation results. Examples of the output device 108 include a display, a printer, and a speaker.

The main storage unit 105 and/or the auxiliary storage unit 107 may store a program. The calculation unit 106 may execute the program, causing the quality management system 101, which is a computer, to execute the functions described in the embodiment. In other words, the program causes the computer to function as the information processing device according to the embodiment. The main storage unit 105 and/or the auxiliary storage unit 107 may be, for example, a memory, a read only memory (ROM), a random access memory (RAM), or a hard disk drive (HDD).

The input units 102, 103, and 104 each have a different function according to a different data take-in method. The input unit 102 takes in the data by linking with other systems. Examples of other systems include a manufacturing execution system (MES), a material management system, an electronic medical record, a patient registry, and a laboratory information management system (LIMS). The input unit 102 accesses a database (DB) 110 of each system and takes in referred data.

The input unit 103 takes in data by linking with a production facility, a monitoring device, or the like. Examples of the production facility and the monitoring device include an automatic culture device for automatically culturing cells, a cell observation system, a cleanliness monitoring device for monitoring the number of floating fungi, the number of parties, and the like in a production environment, a monitoring system for monitoring operation contents performed manually, a movement of an operator in the production facility, and the like, and a transport monitoring device for measuring a temperature, a pressure, and the like during transport.

The input unit 103 accesses a production facility and monitoring device 111 (at least one of the production facility and the monitoring device) and takes in data. The data obtained by the production facility and monitoring device 111 is temporarily stored in certain system, and the input unit 102 is used to refer to the database. It is assumed that the input unit 103 directly takes in data from the production facility and monitoring device 111 without using the database.

The input unit 104 takes in manually input data. As an example, in the regenerative medicine, there may be a process that is manually operated by an operator according to an operation instruction 112. In particular, in such a case, an operation result obtained by manually operation and a monitoring result measured during the operation may be manually entered into the operation instruction 112. The input unit 104 is a data take-in method assuming that the operator or the like manually inputs the contents described in the operation instruction 112 via an input terminal 113 after the operation is completed. Data for the operation instruction 112 may be directly input via the input unit 103. Examples of the input unit 104 may include a keyboard, a mouse, a touch panel, a numeric keypad, a scanner, a microphone, and a sensor.

In addition, an operation result and a monitoring result measured during operation may be recorded on an electronic terminal. In this case, the data may be directly input by the input unit 103 or may be temporarily stored in the database and then input from the input unit 102. The data to be taken in may be generated not only during commercial production after production and marketing approval is obtained, but also throughout the entire life cycle of the cell-processed product, including clinical trials, clinical research, and basic research. As an amount of information increases, accuracy of analysis is considered to increase accordingly. However, in this case, it is sufficiently assumed that a type, an amount, and/or quality of information are different depending on a time of development, and it is preferable to handle data in consideration of this.

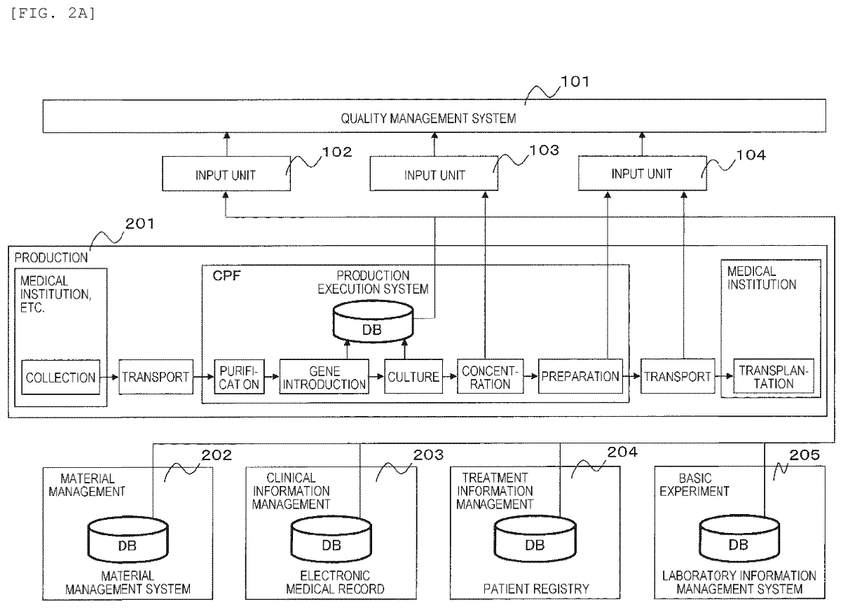

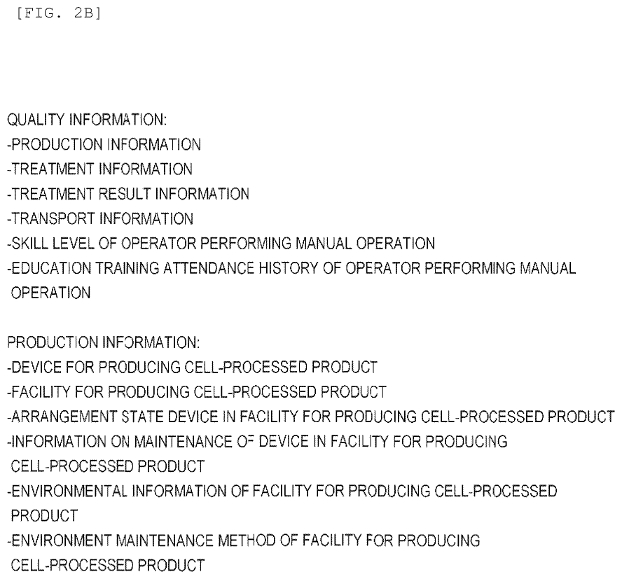

FIG. 2A shows an example of generation of various kinds of information (quality information) in a life cycle of a cell-processed product or the like as data related to production of a cell-processed product or the like accumulated and/or managed by a quality management system, material management of a raw material or the like, clinical information management, treatment information management of adverse events and/or safety information after transplantation, and a basic experiment.

A production process 201 may vary depending on the type of a cell-processed product to be produced or the type of a disease to be treated. Here, processes of collection, purification, gene introduction, culture, concentration, preparation, and transplantation are described. A transport process may be included.

Data related to a material management system in the material management 202, an electronic medical record in clinical information management 203, a patient registry in treatment information management 204, and a basic experiment 205 is accumulated in a laboratory information management system, and other method may be used.

Although the three types of input units 102 to 104 are described with reference to FIG. 1, as shown in FIG. 2A, in the production of a cell-processed product or the like, an execution location may vary depending on the process. For example, a collection process can be performed in a medical institution or the like. Purification, gene introduction, culture, concentration, and formulation are generally performed by CPF. The type of the input unit to be used may be changed depending on the execution location. In addition, the type of the input unit to be used may be changed for each process or for each subdivided operation content even in a process performed in the same CPF.

For example, when an automatic culture device is used in a culture process, data may be directly input to the quality management system from the input unit 103 shown in FIG. 1, or data may be input to the quality management system from the input unit 102 after the data is input to the database of the manufacturing execution system. Alternatively, when an operator manually performs a culture process in a safety cabinet and manually writes an operation result or the like in the operation instruction, data may be manually input using the input unit 104. Further, when production, transplantation, and the like are performed in multiple facilities, a method for generating operation contents and data may change for each facility, and thus an input method to be used may also change. FIG. 2A shows, as an example, a case in which the input unit 102 is used in the gene introduction and the culture process, the input unit 103 is used in a concentration process, and the input unit 104 is used in formulation and transport process.

Various kinds of information in the life cycle of a cell-processed r the like accumulated and/or managed by the quality management system 101 will be described.

FIG. 2B shows an example of the quality information. The quality information includes a plurality of parameters related to a plurality of cell-processed products, and particularly includes a parameter related to at least one of production information, treatment information, treatment result information, and transport information related to the cell-processed products.

The quality information may include a skill level of an operator who performs a manual operation and/or an education training attendance history of the operator who performs the manual operation. In this way, it is possible to perform quality evaluation in consideration of the skill of the operator.

The production information may include at least one of a device for producing the cell-processed product, a facility for producing the cell-processed product, an arrangement state of a device in the facility for producing the cell-processed product, information on maintenance of the device in the facility for producing the cell-processed product, environmental information (for example, cleanliness) of the facility for producing the cell-processed product, and an environment maintenance method (for example, a cleaning method) of the facility for producing the cell-processed product. In this way, it is possible to handle various kinds of information on the cell-processed product. Of course, the quality information may include information other than the above information. Another example will be described below.

The production information is divided into a case in which the production information is manually performed and a case in which the production information is cultured by automated production facility for each process or for each subdivided operation content. The type and/or the number of generated data may differ depending on which is used. In general, the operation content is not numerically designated and the operation result is not recorded in detail in the manual operation rather than an operation performed by the automated production facility. However, regardless of whether it is manual or automated production facility, all pieces of data are accumulated so that the data can be used for subsequent analysis.

Examples of the quality information generated in a cell seeding operation of the culture process include the cell count when the cells to be seeded are collected, a cell survival rate, and a specific protein expression level, as examples of production information. For a culture container to be used for culture, in addition to the type (a shape, a type of a base material, and a culture method), a manufacturer name, a lot number, a production date, an expiration date, and the like are also used as additional information, but data may be accumulated in the material management system as the material management.

Examples of the production information at the time of seeding in the culture container include a type of solvent of a cell suspension at the time of seeding, an amount of the solvent, a composition of the solvent, a cell seeding density, an amount of a medium during culture, a composition of the medium, a liquid feeding speed at the time of seeding, a liquid feeding location, a cell distribution in the culture container after seeding, a shear stress generated on the cells, and a total operation time.

Examples of the transport information include a transport speed, vibration during transport, and a total transport time when the culture container is transported from an operation location such as a safety cabinet to an incubator for culturing after seeding. A temperature and/or a pressure during transport may be included.

These items can have more or less influence on the quality of the produced cell-processed product or the like and the quality of an intermediate, and the quality management system evaluates a magnitude of the influence and a manner of the influence. For example, when the cell survival rate at the time of collection is low, it can be assumed that the activity of the cells is low in the subsequent culture. When the liquid feeding speed at the time of seeding is too high, the magnitude of the shear stress received by the cells also increases, which may affect the subsequent cell growth.

When seeding cells, a temperature and a gas phase in a safety cabinet or the like are generally not controlled as compared to an incubator for culturing, and a temperature decrease and/or a change in the pH of the medium may occur according to the operation time of seeding, which may affect the cells. When transporting from an operation location such as a safety cabinet to an incubator for culturing, a vibration during transport gives vibration and/or impact to the cells. It is also conceivable that a force is generated on the cells due to an acceleration or a path caused by a change in the transport speed, and a cell distribution in the culture container changes, thereby affecting a subsequent culture result. These pieces of information may be included in the transport information.

A total transport time can affect a temperature change and a pH change of the medium, similarly to an operation time of seeding. A range to be controlled and items to be monitored are often different between the operation performed by the automated production facility and the manual operation due to cost and/or labor. It is sufficiently conceivable to expand a control range or increase the number of monitoring items in the future and increase the number and/or types of data so that the analysis of the quality management system has higher accuracy. These pieces of information may be included in the transport information.

In particular, assuming a case of production and/or transplantation in multiple facilities, such information is also important so that the quality management system can analyze a case in which a difference between facilities may affect the quality. Examples for a device to be used include a manufacturer name, a model number, maintenance information (an execution date, a frequency, and a maintenance content), and an initialization method each time the device is used. These pieces of information may be included in the production information.

Regarding a manual operation process performed by the operator, a skill level and an education history for each operator generally differ between facilities. In addition, even for the same operator, the operation content and/or the operation result may be different every time the operation is performed. Therefore, information such as an operator number, the skill level, and the education history associated with an operator name is also important. Regarding a layout of a cell preparation chamber, information such as the number of devices, a distance between devices, and a path is also important. These pieces of information may be included in the quality information.

For example, when the operator transports the culture container from the safety cabinet to the incubator, the temperature and/or the gas phase are not controlled in the space, and thus the temperature decrease and the pH change may affect the cells. Therefore, for example, the transport time may affect the quality. Regarding the production environment, information such as a temperature of the cell preparation chamber and the like in the CPF, cleanliness, a use method related to an aseptic operation of the operator and the like, a cleaning method and a cleaning frequency of the cell preparation chamber and the like, and an entrance and exit method for the operator, such as gowning, is also important. These pieces of information may be included in the quality information.

In donor information on a person who provides a tissue to be collected, information on the donor and the like are accumulated. In FIG. 2A, the information is associated with the collection process. Specific examples of the donor information include a registration date of a donor, an informed consent acquisition date, a registration ID, an age, a biological sample donation history, the cell count collected at the time of latest donation, a medical history such as an infectious disease, a height, a weight, a travel history, results of a blood test and a serological test, and the like. These pieces of information may be included in the quality information.

Data that can be accumulated in the material management system as the material management includes a manufacturer name, a lot number, a production date, an expiration date, a type (a shape and/or a type of a base material), and the like regarding a material to be used. Information to be managed when purchasing the material is assumed. These pieces of information may be included in the quality information.

Regarding the lot number, in particular, a lot number of the serum to be used in the culture greatly affects a culture result. Specific material management information is assumed to include an order number, an order person, an order date, a product name, a manufacturer, an item, a delivery date, a purchase amount, a delivery destination information, an expiration date, and the like. In the case of samples derived from living organisms, an animal species, a part to be used, and a process of use are further added. Depending on the material, it is expected that the material will be divided and used by dispensing and the like, and thus information such as an opening date, a dispensing container, number of dispensed bottles, a dispensing volume, and a branch number is also generated, but these may be treated as the production information. This is because information on an operation date, an operator, and an operation result when an operation such as dispensing is performed is also associated. These pieces of information may be included in the quality information.

Data that can be accumulated in the electronic medical record as clinical information management includes basic information, clinical information, and the like of a patient to be transplanted. It is also assumed that the data may overlap data that can be accumulated in the patient registry as the treatment information management to be described later, and it is preferable to perform adjustment. Specific clinical information is assumed to include a medical institution name, a target disease patient ID, a consent acquisition date, a date of birth, a gender, a height, a weight, an original disease, a medical history, a complication, an allergy, a transplantation date or a transplantation start date, and a transplantation end date. These pieces of information may be included in the quality information.

Among the quality information, data that can be accumulated in the patient registry as the treatment information management includes the treatment information and the treatment result information.

Specific treatment information includes transplanted cell information, raw material information, production process flow information, a transplantation date and time, an administration amount, a person in charge of administration, effectiveness information (whether complete efficacy is achieved at a specific date and time after administration, and survival status), and the like.

Examples of the treatment result information is assumed to include effectiveness after transplantation, an adverse event, safety information, and adverse event information (presence or absence of occurrence of adverse event, total of all adverse events, a name of adverse event, a date of expression, seriousness, a treatment for adverse event, a transfer date, a causal relationship evaluation). The adverse event information is assumed to include more detailed data such as infectious/parasitic diseases, benign/malignant/unspecified neoplasms, blood/lymphatic system disorders, immune system disorders, endocrine system disorders, metabolic/nutritional disorders, mental disorders, nervous system disorders, eye disorders, ear/labyrinth disorders, cardiac disorders, vascular disorders, respiratory/thoracic/mediastinal disorders, gastrointestinal disorders, hepatobiliary system disorders, skin/subcutaneous tissue disorders, musculoskeletal/connective tissue disorders, renal/urinary tract disorders, reproductive system/breast disorders, and congenital/familial/genetic disorders.

Data regarding the basic experiment that can be accumulated in the laboratory information management system may include data acquired as a basic research at an initial stage of development, data that supports the effectiveness of treatment through the addition of more detailed experiments as development progresses and a treatment mechanism becomes clear, and data acquired as a basic research when an opinion differs from the hypothesis. Specific basic experiment information is assumed to include an experiment date, an experimenter name, an experimenter ID, an experiment name, an experiment time, an experiment completion time, a used cell ID, a used cell type name, a cell count, and the like. These pieces of information may be included in the quality information.

The type and amount of data to be acquired may vary depending on an experiment content. For example, in the case of a cell morphology and cell growth evaluation experiment, a cell image, an image ID, an imaging time, a culture container No., an imaging location in the culture container, a morphology evaluation result, details at the time of morphological abnormality, a cell count/cell occupancy, and the like are assumed. In the case of a differentiation ability evaluation experiment, a culture container No., a differentiation induction destination, a cell image, an image ID, a cell morphology evaluation result, a flow cytometry measurement result, a marker expression evaluation result, and the like are assumed. These pieces of information may be included in the quality information.

The data accumulated in the quality management system is associated first. The data of each process is associated with the date and/or the operator ID, and the like, and consistent data in units of lots is created in various kinds of information on the life cycle from collection to transplantation. In particular, association is important for information stored in a system different from the quality management system.

A data format is changed as necessary. When data stored in a system different from the quality management system is accumulated, the specifications of the quality management system are matched. For example, when liquid feeding amounts are indicated in different units such as “10 ml” and “10 cc”, the liquid feeding amounts are unified. Basic statistical information is obtained for all pieces of data. For numerical data, maximum/minimum values, averages, variances, distribution maps, and the like are obtained. Category data is scored and examined in the same manner. In a scoring method, validity is appropriately evaluated.

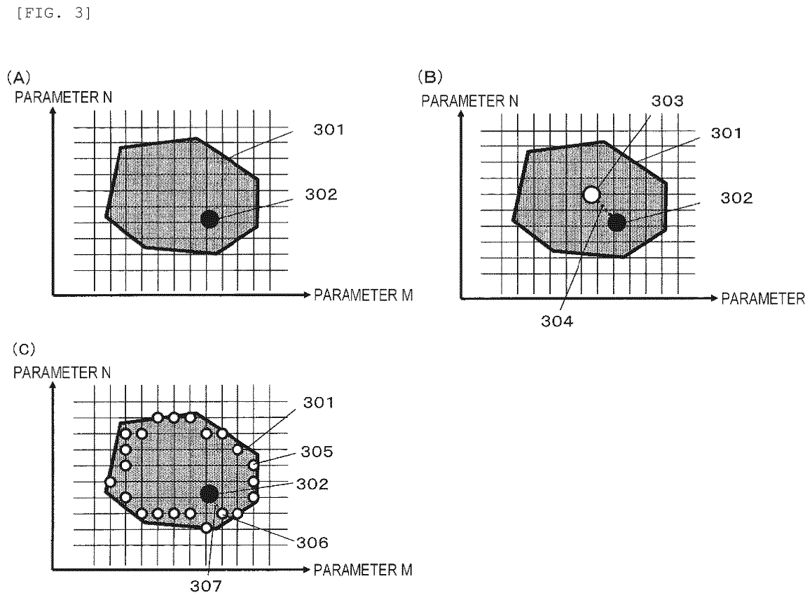

With reference to FIG. 3, a calculation method for digitizing, as product stability, a distance between the center of gravity and/or the boundary of the design space and a point plotted as quality when produced by a certain parameter at a certain time will be described.

As described above, the design space is an interaction with a multi-dimensional combination of input variables (the COA and the like) of a production process and the like which are proven to secure quality.

The design space represents, for example, a specific region (a first region) in a parameter space constituted by parameters of the quality information, and is calculated based on a predetermined quality standard separately input. In particular, the design space corresponds to the first region when the cell-processed product is completed. The quality standard represents a preferable value or range of at least one of the parameters included in the quality information.

The quality standard is stored in the main storage unit 105 and/or the auxiliary storage unit 107 together with the quality information.

If a plotted point for a certain lot during production is within the design space, it can be assumed that the quality is secured. In addition, even when an input variable of the production process or the like is changed, if the plotted point for the lot continues to be within the design space, it can be assumed that quality is secured for that production method. Accordingly, as long as the plotted point for the lot of the selected production method continues to be present inside the design space, the production can be performed on any scale and/or lot.

Here, it can be considered that the quality is secured as long as the plotted point for the lot of the selected production method continues to be present inside the design space, whereas when data obtained in the subsequent production or research and development is newly analyzed, there is a possibility that a range and/or a shape of the design space for securing the quality is changed. In consideration of such a possibility, it is considered that the quality is stable when the plotted point for the lot of the selected production method is located inside the design space at the time of performing the evaluation, and further, when the point is located farther from the boundary inside the design space.

When the range and/or the shape of the design space is changed in the subsequent analysis, it is evaluated that a point plotted as quality in a case of being produced by a certain parameter at a certain time is prevented from being located outside the design space by a relationship of the center of gravity and/or the boundary of the design space.

Regarding the former, it is considered that the point plotted as the quality when produced by a certain parameter at a certain time is located further inside the design space, and the quality is more stable as the distance from the center of gravity of the design space is shorter. Regarding the latter, it is considered that the point plotted as the quality when produced by a certain parameter at a certain time is located farther from the boundary inside the design space, and the quality is more stable as the distance to the boundary of the design space is longer.

Based on the above, both the distance from the center of gravity of the design space and the distance from the boundary of the design space are used as an index of product stability (quality state). As a modification, only one of these may be used as the index of the quality state.

A method for calculating the distance to the center of gravity and/or the boundary of the design space as the quality stability at a point plotted in the design space as the quality when produced by a certain parameter at a certain time will be described.

(A) of FIG. 3 shows a design space 301 and a product position 302 representing a cell-processed product plotted as the quality when produced by a certain parameter at a certain time. The design space 301 is a multi-dimensional combination and interaction of input variables (COA and the like) of a production process and the like for which it is proved that the quality is secured, and if a point plotted for a certain lot during production is within the design space, it can be assumed that the quality is secured.

In (A) of FIG. 3, a region surrounded by the design space 301 (a gray portion in (A) of FIG. 3) is a region where the quality is secured. Although (A) of FIG. 3 shows parameters N and M in two dimensions, the design space is generally more multi-dimensional. There are a case in which it is considered that the quality can be secured as long as the point is within a region surrounded by the design space and a case in which a probability of securing the quality is set at each point of the design space. In the latter case, it is a probabilistic design space. In order to satisfy both methods, coordinates of the design space 301 and the probability of securing the quality at that point are expressed as follows.

Coordinates: (xₘ₁, xₘ₂, xₘ₃, . . . , xₘᵢ, . . . , xₘₙ)

Probability of securing the quality at that point: DS %ₘ

Here, m is an index of a product, i is an index of a parameter, and i=1, 2, 3, . . . , n.

The probability DS %ₘ of securing the quality is set at each coordinate of n-dimensional coordinates (xₘ₁, xₘ₂, xₘ₃, . . . , xₘᵢ, . . . , xₘₙ). When it is considered that the quality can be secured as long as the point is within the region surrounded by the design space 301, a value that the DS % can take is 0 or 100. If there is a product position plotted for a certain lot during production in a region of DS %=100 (the gray portion in (A) of FIG. 3), the quality is secured.

On the other hand, when the probability of securing the quality is set at each point of the design space 301, the value that the DS % can take is between 0 and 100. A magnitude of the value of DS % of each point is the probability that the quality of the plotted point is secured for a certain lot during production. As the value of DS % increases, the probability that the quality is secured increases.

The information on the design space is input in advance from outside of the quality management system. In (A) of FIG. 3, each point constituting the inside and outside of the design space 301 is expressed as a grid point at which each axis intersects. If an interval of each axis is increased, the number of grid points constituting the inside and outside of the design space increases, and thus accuracy also increases, but a load and a processing time in the calculation increase. The accuracy of the points constituting the inside and outside of the design space 301 is determined according to required calculation accuracy.

In the space in which the design space 301 is set, there is the product position 302 (here, a point representing an A-th product is denoted by X_{Ai}) that is an evaluation target and is plotted as the quality when produced by a certain parameter at a certain time. The coordinates of X_{Ai} are as follows.

$${X}_{Ai} = ( {x}_{A 1} , {x}_{A 2} , {x}_{A 3} , … , {x}_{Ai} , … , {x}_{A n} )$$

A calculation method for obtaining a center of gravity 303 of the design space will be described using (B) of FIG. 3. Coordinates X_{Gi} of the center of gravity 303 of the design space

$${X}_{G i} = ( {x}_{G 1} , {x}_{G 2} , {x}_{G 3} , … , {x}_{G i} , … , {x}_{G n} )$$

are obtained by the following formula.

$${X}_{Gi} = [ Σ ( {x}_{m i} * DS {\%}_{m} ) / Σ ( DS {\%}_{m} ) ]$$

The coordinates may be obtained by integration.

$${X}_{Gi} = \int ( {x}_{m i} * DS {\%}_{m} ) d x / \int ( DS {\%}_{m} ) d x$$

In this way, the calculation unit 106 calculates the design space based on the quality standard.

A square distance d (X_{Gi}, X_{Ai}) between the center of gravity X_{Gi} of the design space and the product position 302 (X_{Ai}) plotted for a certain lot during production is defined as a center-of-gravity distance 304.

$$d ( {X}_{G i} , {X}_{Ai} ) = {( {x}_{A 1} - {x}_{G 1} )}^{2} + {( {x}_{A 2} - {x}_{G 2} )}^{2} + {( {x}_{A 3} - {x}_{G 3} )}^{2} + … + {( {x}_{Ai} - {x}_{G i} )}^{2} + … + {( {x}_{An} - {x}_{G n} )}^{2}$$

Next, a calculation method for obtaining the boundary of the design space will be described with reference to (C) of FIG. 3. In the design space 301, coordinates of a point where DS %=100 or coordinates of a point where DS % is close to 100 are obtained. A grid point 305 in the vicinity that is in contact with the inside of the boundary of the design space 301 is obtained. The “grid point in contact with the inside of the boundary” refers to, for example, a grid point in which any grid point adjacent to the grid point is outside the design space 301 among grid points inside the design space 301.

The coordinates of an m-th grid point X_{Bm} in contact with the inside of the boundary are expressed as follows.

$${X}_{B m} = ( {x}_{B m 1} , {x}_{B m 2} , {x}_{B m 3} , … , {x}_{B m i} , … , {x}_{B m n} )$$

Subsequently, the square distance d (X_{Bmi}, X_{Ai}) between the m-th grid point X_{Bm} and the product position X_{Ai} that is plotted as the quality of the product when produced by a certain parameter at a certain time to be evaluated is defined as the distance between the m-th grid point X_{Bm} and the product position X_{Ai}.

$$d ( {X}_{B m i} , {X}_{Ai} ) = {( {x}_{A 1} - {x}_{Bm 1} )}^{2} + {( {x}_{A 2} - {x}_{B m 2} )}^{2} + {( {x}_{A 3} - {x}_{B m 3} )}^{2} + … + {( {x}_{Ai} - {x}_{B m i} )}^{2} + … + {( {x}_{An} - {x}_{B m n} )}^{2}$$

Among the m-th grid points X_{Bm}, a grid point having the smallest square distance d(X_{Bmi}, X_{Ai}) from the product position X_{Ai} is defined as a minimum distance boundary grid point 306, and a square distance dₘᵢₙ (X_{Bmi}, X_{Ai}) between the minimum distance boundary grid point 306 and the product position X_{Ai} is defined as a boundary distance 307 (a minimum distance). When the product position X_{Ai} is outside the design space, the square distance dₘᵢₙ (X_{Bmi}, X_{Ai}) is multiplied by −1 and expressed as a negative value. When the square distance dₘᵢₙ (X_{Bmi}, X_{Ai}) is 0, it is assumed to be on the boundary of the design space.

The center-of-gravity distance 304 and the boundary distance 307 in the design space obtained in this manner are used for evaluation as quality stability indicating the quality state. The smaller the center-of-gravity distance 304 and/or the larger the boundary distance 307, the better the quality.

As described above, the calculation unit 106 calculates the quality state of the cell-processed product based on the design space 301 and the quality information. For example, the calculation unit 106 calculates the center of gravity 303 of the design space 301, and calculates the product position 302 representing the cell-processed product with respect to the design space 301 based on the quality information. Then, the calculation unit 106 calculates the quality state based on the center-of-gravity distance 304 between the center of gravity 303 and the product position 302 (the center-of-gravity distance 304 may be used as the quality state as it is). In this way, the center of gravity 303 representing preferable quality can be used as a calculation standard of the quality state, and the quality of the cell-processed product can be appropriately evaluated.

For example, the calculation unit 106 calculates a boundary of the design space 301 (represented by a grid point in contact with the inside of the boundary in the embodiment), and calculates the product position 302 for the design space 301 based on the quality information. Then, the calculation unit 106 calculates the quality state based on the boundary distance 307 between the boundary and the product position 302 (the boundary distance 307 may be used as the quality state as it is). In this way, the distance from the boundary representing undesirable quality can be used as the calculation standard of the quality state, and the quality of the cell-processed product can be appropriately evaluated.

Here, the square distance is used as an example of distance, but when determining the distance to the center of gravity of the design space in particular, the distance may be determined as a Euclidean distance or a Mahalanobis distance. When the Mahalanobis distance is obtained, the Mahalanobis distance is an evaluation index as a distance in consideration of a variation in each of coordinates constituting the design space.

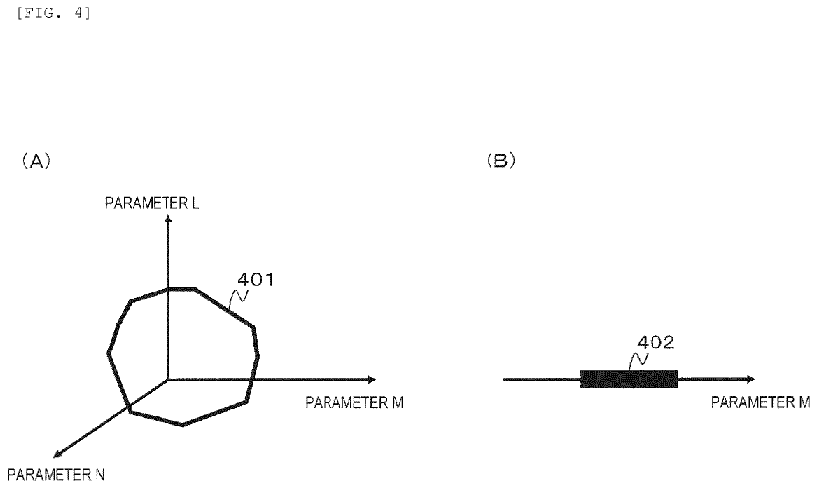

The design space 301 may be a multi-dimensional space. Although FIG. 3 shows a two-dimensional design space as an example, (A) of FIG. 4 shows a three-dimensional design space 401, and (B) of FIG. 4 shows a one-dimensional design space 402. In addition, instead of using a multi-dimensional design space, it is also possible to lower the dimension by performing principal component analysis, which is a statistical method. It is sufficiently assumed that the number of parameters and COAs for constructing the design space is more than three, and in this case, it is multi-dimensional.

A flow of analysis using data accumulated in the quality management system will be described with reference to a diagram of a screen example.

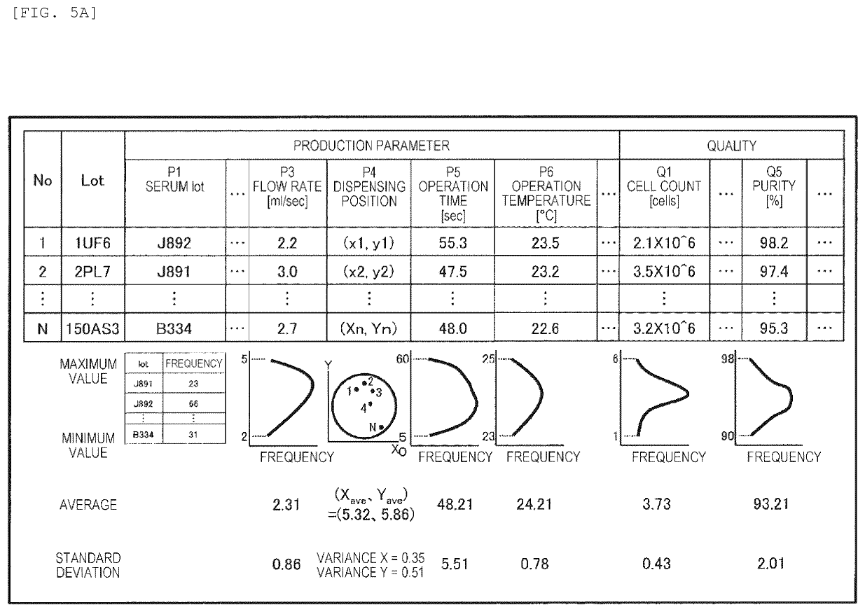

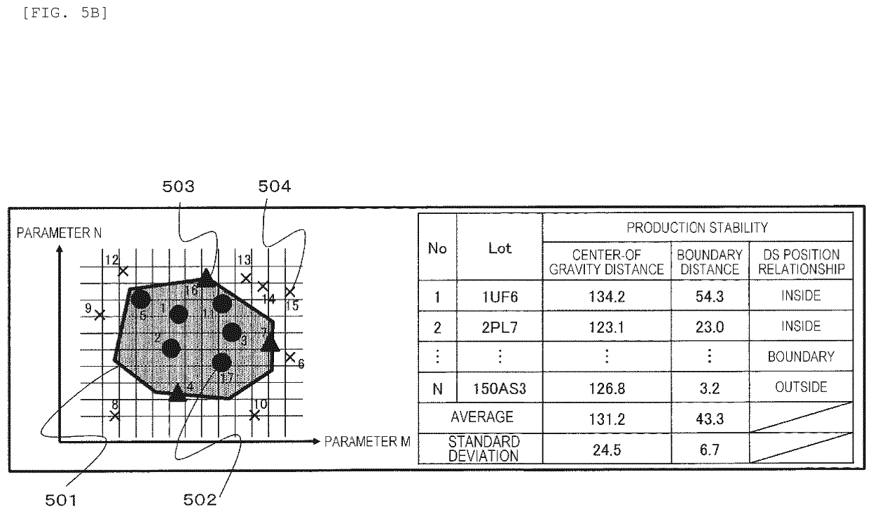

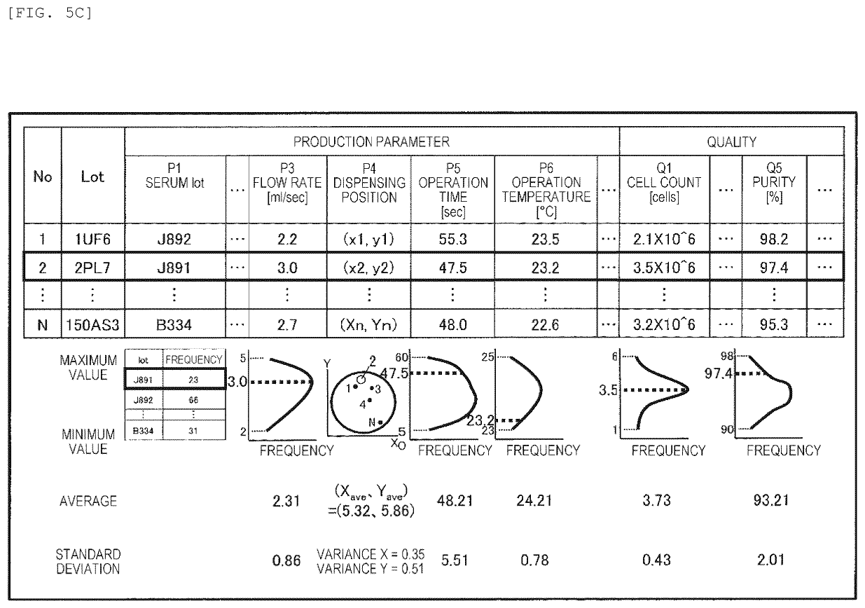

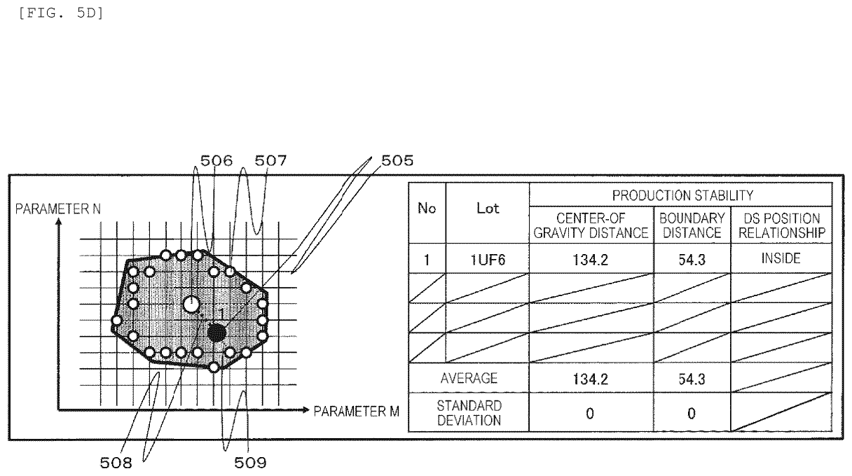

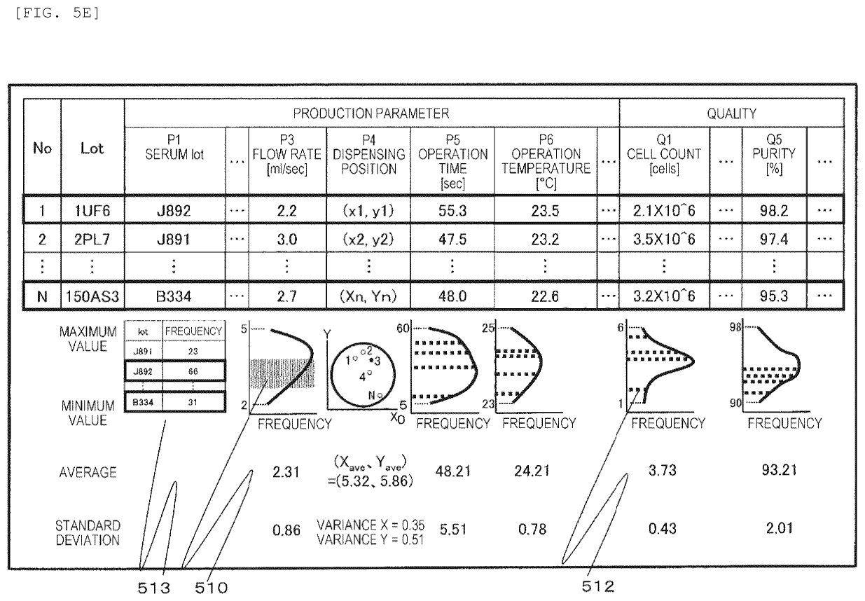

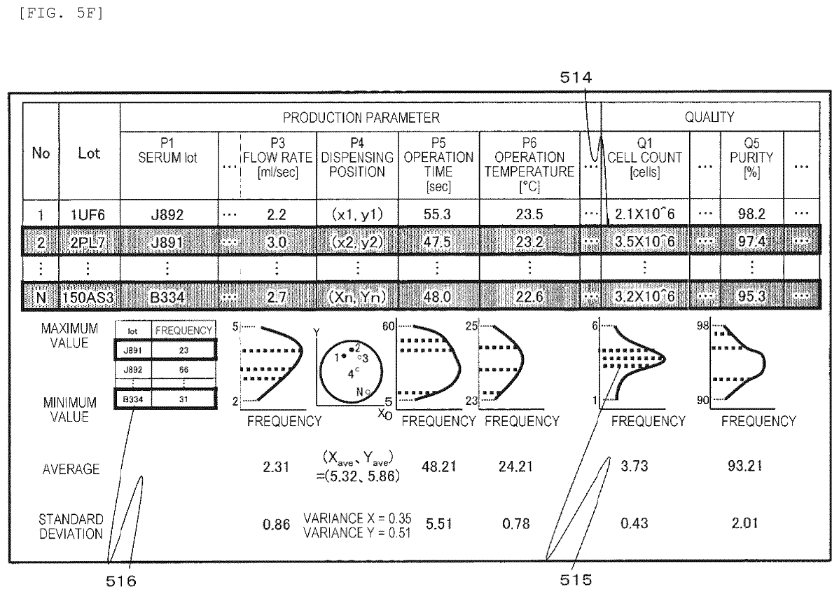

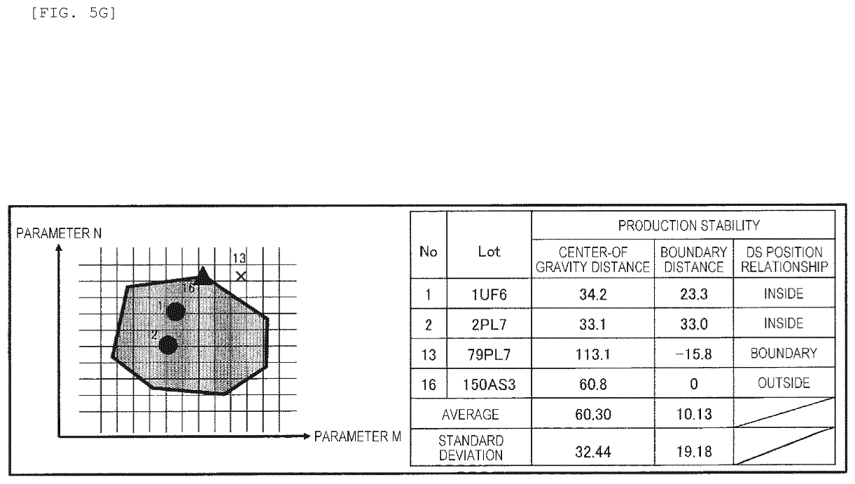

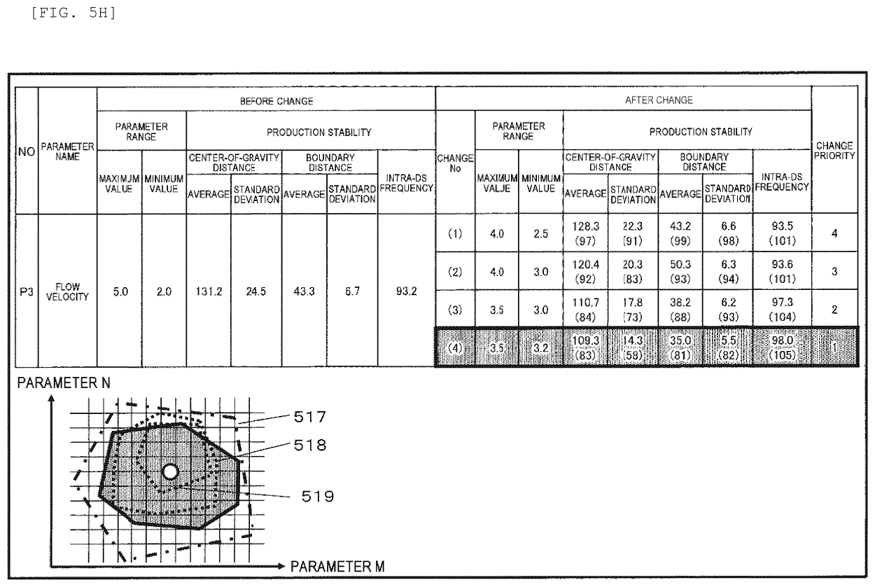

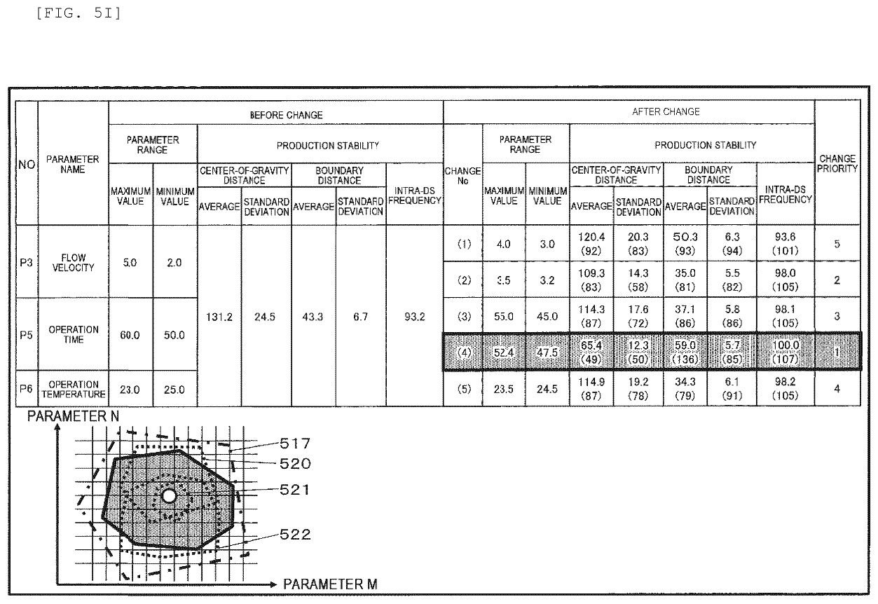

FIG. 5A is a screen that displays various kinds of information on a life cycle of the cell-processed product and the like, such as the production of the cell-processed products, the material management of raw materials and the like, clinical information management, treatment information management such as adverse events after transplantation and/or safety information, and data related to basic experiments, which are accumulated and/or managed by the quality management system.

Both input data for the possibility of changing conditions such as parameters during production, and output data to be analyzed as a result of changing input conditions such as quality after production or prognosis information after transplantation are displayed. When an amount of information is large and the entire information cannot be displayed on one screen, the information is divided and displayed according to a type of information, an occurrence time, and the like.

In the case of numerical data, an average, a standard deviation, a maximum value, a minimum value, a distribution map, and the like are displayed. In the case of category data such as a production location, a serum lot, a used device name, a used device model number, and an operator name, an occurrence frequency of each category is shown in a table. Scoring for assigning a number to each category is performed as necessary, and in the case of the numerical data, the average, the standard deviation, the maximum value, the minimum value, the distribution map, and the like are displayed. In a scoring method, validity is appropriately evaluated. In the case of date data, the latest data is arranged in order from the past like the category data, and the occurrence frequency of each date is shown in a table.

In the case of image data of cells or the like imaged during microscopic observation, a screen displayed in a small size so that images can be listed, a screen in which a desired image is enlarged and displayed as necessary, a screen in which images of the same lot and different imaging dates are arranged, a screen in which images of a plurality of different lots and the same number of culture days are arranged for comparison, and the like is used.

In the case of graph data, similarly to the image data, a screen displayed in a small size so that the graph can be listed, a screen in which a graph desired as necessary is enlarged and displayed, for example, a screen in which graphs of the same lot and different imaging dates are arranged, a screen in which graphs of a plurality of different lots and the same number of culture days are arranged for comparison, and the like are used.

In the case of coordinate data such as coordinates at which a tip of a pipette or the like is located in the culture container when the liquid is fed to the culture container, the coordinates are shown for each lot in the schematic view of the culture container shown by XY coordinates. An average, a variance, and the like at each of the X and Y coordinates are displayed.

In the case of character string data, all pieces of data are displayed in a list. The character string data to be used with high frequency, such as “no abnormality”, “abnormal appearance”, and “culture medium is cloudy” are entered into the quality management system as default data in advance, and whenever such information is entered into the quality management system, any data that can be replaced with the default data is replaced, thereby facilitating analysis.

FIG. 5B shows various types of data to be output, such as quality after production and prognosis information after transplantation, to be analyzed as a result of changing conditions to be input. FIG. 5B is a diagram showing a distribution in a design space 501, and a table showing a center-of-gravity distance and a boundary distance of the design space in each production lot, and a position with respect design space to the (inside/outside/on the boundary). The average and the standard deviation of the center-of-gravity distance and the boundary distance of the design space of each production lot are also displayed.

FIG. 5B also shows a table in which various types of data to be output are displayed on another screen. In the drawing showing the distribution in the design space 501, points corresponding to each production lot are plotted, and product positions 502 in the design space are plotted as circles, product positions 503 at the boundary of the design space are plotted as triangles, and product positions 504 outside the design space are plotted as crosses.

A production lot or a serial number is assigned near each point. By selecting each product position on the screen, the quality information (for example, the production lot, the production date, and the production location) regarding a product corresponding to a selected product position is displayed.

FIGS. 5C and 5D are diagrams in which a certain production lot is selected in the table as shown in FIG. 5C, and a distribution of points of that production lot within the design space as shown in FIG. 5D, as well as tables showing a center-of-gravity distance and a boundary distance of the design space in the production lot, and positions with respect to the design space (inside/outside/on the boundary).

FIG. 5D also shows a product position 505 in the design space of the point of that production lot, a center of gravity 506 of the design space, and a grid point 507 inside the boundary of the design space. A center-of-gravity distance 508 and a boundary distance 509 are also shown. The center of gravity 506 of the design space and the grid point 507 inside the boundary of the design space may be additionally displayed in FIG. 5B.

FIGS. 5E, 5F, and 5G are diagrams in which a plurality of production lots are selected in FIGS. 5E and 5F, diagrams showing the distribution of product positions of the plurality of production lots selected in FIG. 5G within the design space, and tables showing the center-of-gravity distance and the boundary distance of the design space for the production lots, and positions with respect to the design space (inside/outside/on the boundary).

In FIG. 5E, when selecting a part of any input item, a user determines a selection range 510 designated in the graph. Accordingly, the range of the production lot included in the selection range 510 changes from the entire lot (entire range) to a production lot 511 (corresponding range) corresponding to the selection range designated in the graph, and the production lot 511 corresponding to the selection range is highlighted in the table.

Each production lot included in the selection range 510 is displayed in other items as a production lot 512 (corresponding range) in the graph corresponding to the selection range designated in the graph, and a production lot 513 (corresponding range) in the category corresponding to the selection range designated in the graph.

As described above, the calculation unit 106 (FIG. 1) calculates the corresponding range of one or more other types of parameters based on a range designated by one or more types of parameters included in the quality information. The output unit 108 (FIG. 1) outputs the calculated corresponding range.

In FIG. 5F, when selecting a part of any input item, the user determines a selection range 514 (one or more ranges including one or more production lots) designated in the table. The production lots included in the selection range 514 are displayed in other items as a production lot 515 in the graph corresponding to the selection range designated in the table, and a production lot 516 in the category corresponding to the selection range designated in the table.

For a plurality of production lots selected in FIGS. 5E and 5F, a diagram showing the distribution in the design space in FIG. 5G and a table showing the center-of-gravity distance and the boundary distance of the design space in the production lot and the position with respect to the design space (inside/outside/on the boundary) are displayed.

As shown in FIGS. 5E, 5F, and 5G, by setting the range of parameters to be examined with respect to the input information, it is possible to grasp the distribution of the input parameters other than the parameters to be examined and the distribution of the output results. In addition, it is possible to see where the input parameter is located on the graph or the category table. Further, as a result of the output, for example, it can be seen how the quality state in the production lot is located in the design space.

As shown in FIG. 5G, for the result indicating the distribution of the quality state of the production lot in the design space, it is possible to quantitatively grasp a tendency of the quality state of the production lot based on the center-of-gravity distance and the boundary distance of the design space and the position with respect to the design space (inside/outside/on the boundary).

As shown in FIG. 5E, when there is a parameter to be examined, the user determines a selection range 510 designated based on the parameter, and plots the quality state based on the designated range in the design space. In this case, as the set of plotted product positions is closer to the center of gravity of the design space, the production is performed in a range set in the parameter to be examined, so that the risk that the lot produced thereafter is located outside the design space is reduced.

If the product can be produced, for example, in a range set in the parameter to be examined as the product position is farther from the boundary inside the design space, the risk that the lot produced thereafter is located outside the design space is also reduced.

Further, by sequentially examining the parameters, a set of points plotted in the design space may be distributed over a wide range or may be distributed only locally. An average, a median, a standard deviation, a confidence interval, and the like in a set of points plotted in the design space are obtained, a difference is quantitatively obtained while statistically comparing a set of points plotted in the design space obtained when each parameter is examined, and these are also used as materials for understanding a relationship between an input and an output.

In FIGS. 5E, 5F, and 5G, the screen example in which the data to be input is selected and a behavior of the data to be output is displayed is described, and the display may be reversed. That is, the output data is selected, and the behavior of the input data is displayed. For example, in the screen shown in FIG. 5B, data to be examined is selected from a design space diagram on the left side of the screen or a table on the right side of the screen. Then, features of data such as parameters corresponding to the data in the selected design space are displayed as shown in FIGS. 5E and 5F. These also help to grasp a relationship between data.

FIG. 5H is a display screen showing values before and after the change of the center-of-gravity distance and the boundary distance of the design space of the production lot, which are the output, as a result of making a plurality of changes to a single type of parameter to be examined.

For example, it is assumed that the distribution of a “flow velocity”, which is one of the parameters, is in a range of 2.0 to 5.0 in all products. The user of the quality management system 101 designates a changed range of the parameter. In the example of FIG. 5H, four ranges of change Nos. (1) to (4) are designated.

The calculation unit 106 (FIG. 1) calculates each quality state based on the plurality of ranges designated by the parameter to be changed. In the example of FIG. 5H, the average of the center-of-gravity distance, the standard deviation of the center-of-gravity distance, the average of the boundary distance, the standard deviation of the boundary distance, and a DS frequency (a frequency of the products whose product positions are present in the design space among the products included in the range) are calculated and output. In relation to the value after the change, a ratio to the value before the change (shown in parentheses in FIG. 5H) is also displayed.

The range of the product position of each production lot with respect to the design space is shown before and after the change. In the example of FIG. 5H, a positional relationship between a set 517 of production lots before the change, a set 518 of production lots corresponding to the change No. (2), and a set 519 of production lots corresponding to the change No. (4) is visualized.

As described above, the calculation unit 106 calculates each quality state based on a plurality of ranges designated by one or more types of parameters. The output unit 108 outputs the calculated quality state.

Among the sets of production lots before and after the change, the center-of-gravity distance and the boundary distance of the design space are used as indices, and a set that is present at the center of the design space is considered to be more stable, and a priority for changing the process or the like is given and displayed. The user examines the change of the production method for the high priority. If the position of the set of production lots hardly changes with respect to the design space even if the parameters are changed, it is determined that there is little significance in examining the change.

FIG. 5I is a display screen showing values before and after the change of the center-of-gravity distance and the boundary distance of the design space of the production lot, which are the output, as a result of making one or more changes to each of a plurality of types of parameters to be examined.

For example, it is assumed that the distribution of the “flow velocity”, which is one of the parameters, is in a range of 2.0 to 5.0 in all products. The user of the quality management system 101 designates a changed range of the parameter. In the example of FIG. 5I, two ranges of change No. (1) and No. (2) are designated for the “flow velocity”. Although not particularly shown in FIG. 5I, information indicating which parameter is changed (in this case, the “flow velocity”) may be displayed.

In the example of FIG. 5I, two ranges of change No. (3) and No. (4) are designated for the “operation time”. Further, in the example of FIG. 5I, one range of the change No. (5) is designated for the “operation temperature”.

In the example of FIG. 5I, the positional relationship between the set 517 of the production lots before the change, a set 520 of the production lots corresponding to the change No. (2), a set 521 of the production lots corresponding to the change No. (4), and a set 522 of the production lots corresponding to the change No. (5) is visualized.

For a plurality of parameters to be examined, a method for changing a process in each of the plurality of parameters is grasped, and then, when the process is preferentially performed from any change, it is examined whether a position of a set of production lots with respect to a design space can be changed to a more stable place, and a change in contribution to improvement in quality is examined.

In the examination here, FIG. 5H similarly applies, but the necessity of the change may be examined by including the cost for the change of the process, the required time, the risk associated with the change, and the like as indexes. A method for evaluating the center-of-gravity distance, the boundary distance, and the like of the design space of the production lots as an output as an index and giving priority to improvement for a change in a plurality of parameters to be examined is performed in the same flow as in FIG. 5I.

The calculation unit 106 (FIG. 1) may generate a change priority (change recommendation information) indicating a recommended range for one or more types of parameters based on a plurality of ranges designated for the one or more types of parameters. The output unit 108 may output the generated change priority. For example, in the example of FIG. 5H, the change priority is determined based on an intra-DS frequency for all the sets after the change. The higher the intra-DS frequency, the higher the change priority. Similarly, in the example of FIG. 5I, the change priority is determined based on the intra-DS frequency for all the sets after the change. In the example of FIG. 5I, the change priority is determined using not only the intra-DS frequency but also other information (not particularly described but can be set as appropriate). In this way, by generating the change priority, it is possible to more easily grasp a preferable parameter range.

In the screens shown in FIGS. 5H and 5I, it is also conceivable to particularly extract parameters related to biological samples such as cells and serum, which are raw materials that are difficult to control in regenerative medicine, and determine a control range while comparing the parameters with controllable parameters.

An example of an improvement method in the case of preferentially improving a parameter having a large influence on the quality using a result obtained from a quality management system will be described. In the case of changing a parameter during production, a setting value is changed if the parameter is related to a device being used. When it is difficult to change the device being used, another device having the same function may be used. For example, another device having the same function used in another production facility may be used.

If the parameter is related to the manual operation by the operator, an instruction content described in the operation instruction is changed. It is difficult to change only the instruction content, and in a case in which the operation content and/or the operation result may vary depending on the skill level of the operator, the operation content and/or the operation result is made uniform by performing education training. In particular, although operators and/or education training are often different between facilities, if a possibility that a difference in operators and/or education training affects operation contents and/or operation results is suggested by inputting the information to the quality management system and using the information for analysis, the possibility is examined.

The parameter related to the manual operation of the operator may vary in operation content and/or operation result even for the same operator. This point of view is also examined. When it is necessary to control the operation content and/or the operation result of the parameter related to the manual operation of the operator with higher accuracy, for example, it is also conceivable to make the operation content and/or the operation result uniform by capturing a moving image of an operation scene, quantitatively analyzing the operation content and/or the operation result based on image analysis, and feeding back the result to the operator in the form of education training or the like. When it is concluded that sufficient quality cannot be secured by manual operation contents and/or operation results, an improvement to automate the operation is also conceivable.

Regarding the production environment, the temperature and the cleanliness of the cell preparation chamber and the like in the CPF are always managed, but there are also influences of a use method, a cleaning method, and a cleaning frequency related to an aseptic operation and the like of an operator, an entrance and exit method, such as a gowning, of the operator, and the like. When such information is also input to the quality management system as quality information and used for analysis, if it is suggested that the difference in production environment may affect the quality or the like, the possibility is examined. Although the change in the setting values of the temperature and the cleanliness of the cell preparation chamber or the like is roughly the same as a countermeasure related to parameters manually performed by the operator in production, it is conceivable to change the description of the operation instruction regarding the aseptic operation or the like of the operator, perform education training, and the like. When a safety cabinet is used, wiping and disinfection when a material is put therein, an operation method of a pass box used when a material is moved between rooms, a gowning method in entrance and exit of an operator, and the like are targeted.

When production or the like is performed in multiple facilities, differences may occur in the operation method of the CPF, the operation content of the operator, a layout of the cell preparation chamber, a use method and maintenance management of a device to be used for production, and the like. When such information is also input to the quality management system as the quality information and used for analysis, if it is suggested that the difference between facilities may affect the quality or the like, the possibility is examined. The operation method of the CPF and the examination and improvement of the operation content of the operator are basically the same as the above-described content.

Regarding the layout of the cell preparation chamber, a type, a number, a model number, and maintenance information on the device to be used are used for analysis. For example, even if the same device is used, when maintenance, a periodic initialization method of the device, or the like is different, it is considered that the quality is affected as a result. These pieces of information may be included in the quality information. In addition, even devices having the same function may be affected by a slight difference in specifications.

As an example, an incubator for culturing cells is generally used at a culture temperature of 37° C., but an upper limit temperature and a lower limit temperature when a culture temperature is set to 37° C. may vary depending on manufacturers. In addition, a frequency of opening and closing an incubator may be different for each facility, and a temperature and a gas phase (for example, a carbon dioxide concentration) change when the door is opened. The vibration and the impact caused during opening and closing vary depending on the specifications of a door (cushioning against the vibration and the impact, a weight of the door, a height of a handle of the door, and the like) and a use method by the operator. It is preferable to input these pieces of information to the quality management system as the quality information and analyze the quality information as to whether the difference is to the degree of negligible or non-negligible with respect to the quality.

Regarding the layout of the cell preparation chamber, it is preferable to input a distance between the devices and a route as the quality information to the quality management system and perform the analysis. For example, when the operator manually performs the operation on the culture container in the safety cabinet and transports the culture container to the incubator, a transport time is determined by a walking speed and distance of the operator. In general, the temperature and the gas phase of the culture container are not controlled during the operation time and the transport time in the safety cabinet, and a temperature decrease and a pH change may affect the cells. It is preferable to analyze whether the difference between the environment inside an incubator in which the temperature and the gas phase are controlled and the difference between the environment inside and the incubator is negligible or not negligible with respect to the quality.

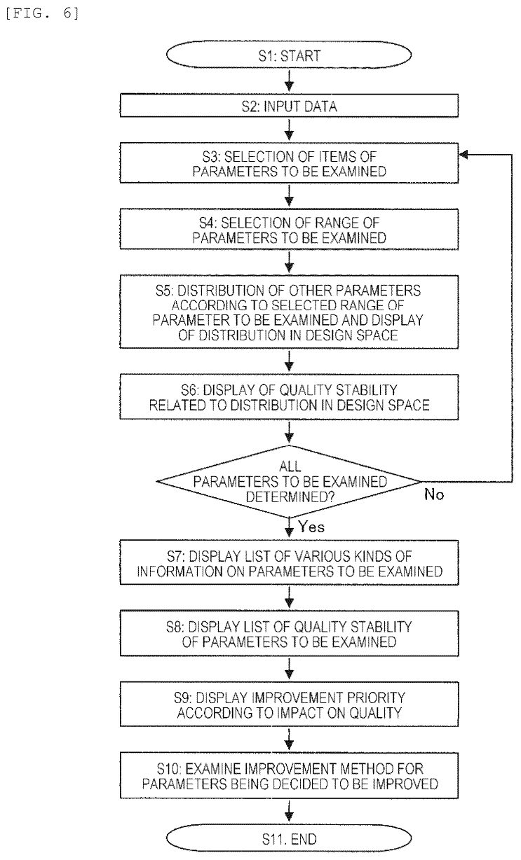

FIG. 6 shows a series of procedures for evaluating the quality and obtaining the priority for improving the process using the quality management system having the above functions.

### <Step S1: Start>

The quality management system is activated.

### <Step S2: Input Data>

Quality information in a life cycle of a cell-processed product or the like, such as each process of collection, purification, gene introduction, culture, concentration, transport, transplantation, and the like, material management of raw materials and the like, and clinical information such as adverse events and/or safety information after transplantation, is input to the quality management system. As shown in FIG. 1, the input method is selected according to the form of the input source data.

After the input, features of various kinds of information are displayed. For example, in the case of numerical data, an average, a standard deviation, a maximum value, a minimum value, and a distribution map are displayed. In the case of category data, an occurrence frequency of each category is shown in a table. The character string data is subjected to processing such as replacement with data stored in advance as default data in the quality management system according to the content, thereby facilitating analysis. In addition, various types of data to be output such as quality after production and prognosis information after transplantation, which are to be analyzed as a result of changing conditions to be input, are displayed.

A diagram showing the distribution within the design space, the center-of-gravity distance and the boundary distance of the design space for each production lot, and the position with respect to the design space (inside/outside/on the boundary) are shown. The average and the standard deviation of the center-of-gravity distance and the boundary distance of the design space of each production lot are also displayed. A table showing all the output data is also provided.

### <Step S3: Selection of Items of Parameters to be Examined>

The user selects and inputs a parameter to be examined as an input condition. The feature of each parameter grasped in step S2 and the feature such as the quality after production and the prognosis information after transplantation to be the output are examined as materials.

### <Step S4: Selection of Range of Parameters to be Examined>

The user selects and designates a range to be examined in the parameter to be examined selected in step S3. As shown in FIGS. 5E and 5F, the selection range may be determined based on a graph or the like showing the distribution of items to be input, or the selection range may be determined based on a table listing various types of data.

### <Step S5: Distribution of Other Parameters According to Selection Range of Parameter to be Examined and Display of Distribution in Design Space>

The system indicates a distribution of other parameters corresponding to the selection range of the parameter to be examined selected in step S3. That is, the corresponding range of one or more other types of parameters is calculated and displayed based on the range designated by one or more types of parameters. The distribution in the design space is shown. For each, a feature corresponding to the selection range is displayed. For example, in the case of the numerical data, an average, a standard deviation, a maximum value, and a minimum value are displayed. In the case of the category data, the occurrence frequency of each category is shown in a table. In step S5, the display as shown in FIG. 5E or 5F is performed.

### <Step S6: Display of Quality Stability Related to Distribution in Design Space>

As the quality stability, the center-of-gravity distance and the boundary distance of the design space in each production lot and the position with respect to the design space (inside/outside/on the boundary) corresponding to the selection range of the parameter to be examined selected in step S3 are displayed. An average, a standard deviation, and the like regarding the center-of-gravity distance and the boundary distance of the design space of each production lot are also displayed. Various types of data to be output are also displayed. By step S6, the display as shown in FIG. 5G is performed.

After step S6, when all of the parameters to be examined are determined, the process proceeds to step S7. When all of the parameters to be examined are not determined, the process returns to step S3 and the examination is performed again. Whether all of the parameters to be examined are determined may be determined based on an input from the user or may be automatically determined.

In the case of automatic determination, when all the calculated product positions are within the design space in the display in step S6, it is determined that all of the parameters to be examined are determined, and otherwise, it is determined that all of the parameters to be examined are not determined. In the case of the automatic determination, a more complicated determination standard using the center-of-gravity distance and/or the boundary distance may be used.

### <Step S7: Display List of Various Kinds of Information on Parameters to be Examined>

In this step, information on various parameters (for example, all of the parameters) to be compared and examined is displayed in a list. As shown in FIG. 5H, a result of evaluating a plurality of selection ranges in one type of parameter may be examined, or a result of evaluating a selection range in a plurality of parameters may be examined. B step S7, the display as shown in FIG. 5E or 5F is performed again.

### <Step S8: Display List of Quality Stability of Parameters to be Examined>

As the quality stability, the center-of-gravity distance and the boundary distance of the design space in each production lot and the position with respect to the design space (inside/outside/on the boundary) corresponding to the selection range of the parameter to be examined selected in step S7 are displayed. An average, a standard deviation, and the like regarding the center-of-gravity distance and the boundary distance of the design space of each production lot are also displayed. All pieces of data to be output are also displayed. By step S8, the display as shown in FIGS. 5H and 5I is performed.

### <Step S9: Display Improvement Priority According to Impact on Quality>

For the center-of-gravity distance and the boundary distance of the design space of the production lot to be output, the change priority is calculated using the values before and after the change, the ratio to the value before the change, and the like. The center-of-gravity distance and the boundary distance of the design space are used as indices, and those existing at the center of the design space are made more stable. In addition to the center-of-gravity distance and the boundary distance of the design space, the cost for the process change, a required time, a risk associated with the change, and the like may be included in the index to calculate the change priority. Calculation results are used and displayed as the priority for changing the process or the like. As described above, the calculation unit 106 (FIG. 1) generates and displays the change recommendation information indicating a recommended range for the one or more types of parameters based on the plurality of ranges designated by the one or more types of parameters. By step S9, the change priorities of FIGS. 5H and 5I are displayed.

### <Step S10: Examine Improvement Method for Parameters Being Decided to be Improved>

A change in the production method is examined for those having a high priority. For example, a degree of importance (a priority) of each parameter in the quality information is stored in advance, and from among sets of parameters after change, a set in which the change in a parameter having a higher degree of importance is smaller is selected, and an improvement is proposed according to the selected set.

More specifically, first, among the sets after the parameter change, all the cell-processed products whose product positions are within the design space are selected. Next, for each of such sets, for a parameter having a high degree of importance (for example, a parameter having the highest degree of importance), a ratio of a width of a range after the change of the parameter to a width of a range before the change is calculated. Then, a set having the highest ratio is selected, and the parameter range of the selected set is output as an improvement proposal.

If the position of the set of the production lots hardly changes with respect to the design space even if the parameters are changed, it is determined that there is little significance in examining the change. For example, information identifying the changed lot set having the highest change priority is displayed.

### <Step S11: End>

When the examination is completed, an examination result is electronically stored in the auxiliary storage unit in a storage. The operation of the quality management system is ended by an appropriate operation.

According to the quality management system implemented as described above, it is possible to generate more useful information on a relationship between the parameters of the cell-processed product. For example, it is possible to visualize a relationship between each parameter related to production or the like serving as an input and the quality or the prognosis information serving as an output. Using the center-of-gravity distance and the boundary distance of the design space digitized as the product stability, it is possible to rank the priority of the items to be improved during production or the like. As a result, it is possible to stabilize the quality of a cell-processed product or the like.

### Embodiment 2

Regarding the quality management system described in Embodiment 1, an embodiment different from Embodiment 1 will be described.

Data related to various kinds of information in a life cycle of a cell-processed product or the like, such as each process of collection, purification, gene introduction, culture, concentration, formulation, transport, transplantation, and the like, material management of raw materials and the like, and clinical information such as adverse events and/or safety information after transplantation, which are obtained up to a certain time, is input to the quality management system as quality information.

In the production after a certain time, data up to an intermediate stage of the production is input. The position of the data up to that point in the design space is calculated and displayed. The center-of-gravity distance and the boundary distance of the design space are obtained as the product stability as well as the positional relationship such as the inside, the outside, and the boundary with respect to the design space, and a positional relationship is quantitatively evaluated.

Using these, the quality at the end of production is predicted based on the data up to the intermediate stage of the production. As the quality standard, the quality standard at the time of completion of the cell-processed product is used as in Embodiment 1. That is, in Embodiment 2, the design space also corresponds to the first region when the cell-processed product is completed. The calculation unit 106 (FIG. 1) determines whether each cell-processed product is acceptable at the time of completion based on the design space and the quality information on each cell-processed product in the intermediate stage of the production.

Those skilled in the art can appropriately design a specific method for predicting data at the time of completion (which may be quality information or may be a product position in a design space) based on the quality information in the intermediate stage of the production. For example, a function for receiving the quality information in the intermediate stage of the production as an input and outputting a product position may be stored in advance. A specific content of the function can be appropriately defined by those skilled in the art based on known techniques and the like. Machine learning can also be used for the specification.

When the product position indicating the quality information on the cell-processed product is inside the design space, the cell-processed product is determined to be acceptable. If the product position is outside the design space, it is determined that the cell-processed product is not acceptable. The output unit 108 may output a pass or fail determination result. An output of a determination result may be a screen as shown in FIG. 5B, for example.

When it is found that the product is located outside the design space with respect to the data up to the intermediate stage of the production, it is conceivable to adopt an option of stopping the production of the product by the designation of the user or automatically by the system. This is because, rather than evaluating the quality at the end of production and determining that a shipping determination standard is not satisfied, the cost can be reduced by predicting that the shipping determination standard is not satisfied in the intermediate stage of the production and stopping production.

In addition, when it is predicted that the shipping determination standard is not satisfied in the intermediate stage of the production, it is conceivable to change the production method so that the shipping determination standard is satisfied at the end of production. However, in this case, it is preferable that a change in the production method during production is recognized during production permission.

### Embodiment 3

Regarding the quality management system described in Embodiment 1, an embodiment different from Embodiment 1 will be described.

Data related to various kinds of information in a life cycle of a cell-processed product or the like, such as each process of collection, purification, gene introduction, culture, concentration, formulation, transport, transplantation, and the like, material management of raw materials and the like, and clinical information such as adverse events and/or safety information after transplantation is input to the quality management system.

In the visualization of each parameter serving as the input and the result serving as the output, the output is any one of the quality at the end of the collection process, the quality at the end of the transport process immediately after the collection process, the quality at the end of the production process, the quality of the intermediate product during the production process, the quality at the end of the transport process immediately after the production process, the quality immediately before or immediately after the end of the transplantation process, and the like.

In Embodiment 3, the calculation unit 106 (FIG. 1) uses the quality information up to a specific process (a first process) as an input, and calculates a quality state at a time when a process (a second process) after the first process is ended based on the quality information. In Embodiments 1 and 2, the first region related to the quality standard is the design space at the time of completion of the cell-processed product, whereas in Embodiment 3, the first region is calculated based on the quality standard at the time of completion of the second process during production.

The first process and the second process can be freely selected in the production process of the cell-processed product. The quality standard at the end of each process can be appropriately defined by those skilled in the art.

In Embodiment 3, the center-of-gravity distance and the boundary distance are calculated not for the design space at the time of completion of the product but for a parameter space during production serving as an output. In addition, the center-of-gravity distance and the boundary distance in the design space at the time of completion may be calculated.

A specific method for calculating the quality state at the time when the second process is completed based on the quality information up to the first process can be appropriately designed by those skilled in the art. For example, a function for receiving quality information up to the first process as an input and outputting a product position in the second process may be stored in advance. The specific content of the function can be appropriately defined by those skilled in the art based on known techniques and the like. Machine learning can also be used for the specification.

The input is basically data before a time at which information on the selected output is generated with respect to the selected output. Data after the time when the information on the selected output is generated is not included in the input. The reason is that an event occurring in the future does not affect the past. However, for example, when the quality at the end of the transport process immediately after the collection process is used as the output, when the evaluation of a cell survival rate or the like serving as the quality at the end of the transport process does not have much influence and a cell growth property or the like when cells purified as the production process are seeded and cultured in a culture container thereafter is affected, the cell growth property or the like is included in the output, and data generated until the time when data on the cell growth property is obtained is included in the input.

The quality at the end of the collection process, the quality at the end of the transport process immediately after the collection process, the quality at the end of the production process, the quality of the intermediate product during the production process, the quality at the end of the transport process immediately after the production process, the quality immediately before or immediately after the end of the transplantation process, and the like, which are set as the output, are set as quality standards.

If the quality standard is satisfied, the process proceeds to the next process, and if the quality standard is not satisfied, the process does not proceed to the next process. A range satisfying the quality y standard is determined using each quality standard. When the quality standard includes one type of parameter, the range satisfying all the quality standards is one-dimensional. When the quality standard includes two types of parameters, the range satisfying all the quality standards is two-dimensional, when the quality standard includes three types of parameters, the range satisfying all the quality standards is three-dimensional, and when the quality standard includes more than three types of parameters, the range satisfying all the quality standards is more dimensions. The range (the first region) satisfying all the quality standards set in this way can be handled like the design space shown in Embodiment 1. An output of a calculation result may be a screen as shown in FIG. 5B, for example.

In the quality management system, the quality at the end of the collection process, the quality at the end of the transport process immediately after the collection process, the quality at the end of the production process, the quality of the intermediate product during the production process, the quality at the end of the transport process immediately r the production process, the quality immediately before or immediately after the end of the transplantation process, and the like are set as outputs. As an input, data before a time at which information on the selected output is generated is input to the selected output.

Depending on the type of the selected output, data generated in the future of the selected output is also input as described above. On the other hand, by using the same method described in Embodiment 1 and changing each parameter which is an input, it is visualized how other types of parameters of the input and information on a selected output which is an output move within a range satisfying all quality standards.

A distance between a point plotted as the quality of a certain parameter and the center-of-gravity distance and the boundary distance of the range satisfying all of the quality standards is digitized as the product stability, and a fluctuation range of the product stability with respect to a fluctuation range of each parameter is also digitized. Based on these values, priorities are ranked as items to be improved in a process up to the selected output. Then, parameters having a large influence on the quality standard are preferentially improved. Details of the improvement are the same as those of Embodiment 1. As a result, it is possible to stabilize the quality of a cell-processed product or the like.

### Embodiment 4

Regarding the quality management system described in Embodiment 1, an embodiment different from Embodiment 1 will be described.

In the flow described in FIG. 6, the results performed by the operator in steps S6 and S9 are accumulated, and a machine learning model for selecting parameters and the like in steps S3, S6, S9, and the like using the results is generated as a prognosis information prediction model.

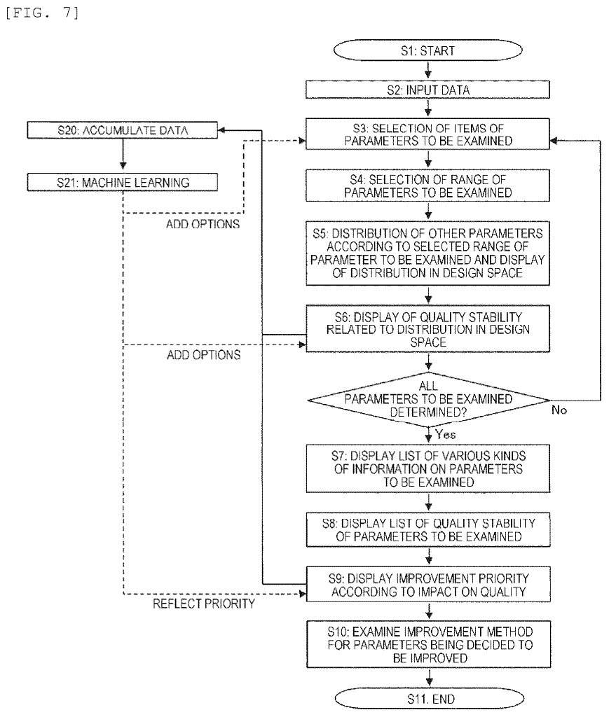

As shown in the flow of FIG. 7, data is accumulated in step S20, the machine learning is performed in step S21, and the result is reflected in steps S3, S6, S9, and the like. As a machine learning model, a well-known or publicly known method such as a neural network or logistic regression may be adopted, and therefore will not be described in detail in the embodiment. In the selection performed by the operator in S3, S6, S9, and the like, the selection by the machine learning is also displayed.

The machine learning can be performed by inputting the parameter range before the change and outputting the parameter range after the change having the highest change priority. For example, in steps S6 and/or S9, it is possible to create training data in which the parameter range before the change is input and the parameter range after the change having the highest change priority is output.

By using such a trained model, in steps S3, S6, S9, and the like, a suitable parameter range after the change can be calculated based on the parameter range before the change. The calculated range may be reflected as a range having the highest change priority in step S9. In this way, the change recommendation information is output.

Accordingly, accuracy of an examined content is improved, and as a result, quality stabilization of a cell-processed product or the like can be implemented. Further, it is possible to implement product production in consideration of enormous types of parameters related to the cell-processed product.

### REFERENCE SIGNS LIST

101: quality management system (information processing device)
102 to 104: input unit (input device)
105: main storage unit (storage device)
106: calculation unit (processor)
107: auxiliary storage unit (storage device)
108: display unit (output device)
109: display unit (output device)
110: database
111: monitoring device
112: operation instruction
113: input terminal
201: process
202: material management
203: clinical information management
204: treatment information management
205: basic experiment
301: design space
302: product position
303: center of gravity
304: center-of-gravity distance
305: grid point
306: minimum distance boundary grid point
307: boundary distance (minimum distance between boundary and product position)
401, 402: design space
501: design space
502 to 505: product position
506: center of gravity
507: grid point
508: center-of-gravity distance
509: boundary distance
510: selection range
511 to 513: production lot
514: selection range
515, 516: production lot
517 to 522: set of production lots

1. An information processing device comprising:
an input device;
an output device;
a processor; and
a storage device, wherein
the input device receives quality information including a plurality of parameters related to a plurality of cell-processed products as an input, the quality information including a parameter related to at least one of production information, treatment information, treatment result information, and transport information related to the cell-processed products,
the storage device stores a predetermined quality standard and the quality information,
the processor
calculates a first region based on the quality standard,
calculates a quality state of the cell-processed products based on the first region and the quality information, and
calculates a corresponding range of one or more types of other parameters based on a range designated by one or more types of parameters, and
the output device outputs the corresponding range.

2. The information processing device according to claim 1, wherein
the processor
calculates a center of gravity of the first region,
calculates, based on the quality information, a product position representing the cell-processed product for the first region, and
calculates the quality state based on a distance between the center of gravity and the product position.

3. The information processing device according to claim 1, wherein
the processor
calculates a boundary of the first region,
calculates, based on the quality information, a product position representing the cell-processed product for the first region, and
calculates the quality state based on a minimum distance between the boundary and the product position.

4. The information processing device according to claim 1, wherein
the quality information includes at least one of
a skill level of an operator who performs a manual operation, and
an education training attendance history of the operator.

5. The information processing device according to claim 1, wherein
the quality information includes the production information, and
the production information includes at least one of
a device that produces the cell-processed product,
a facility that produces the cell-processed product,
an arrangement state of the device in the facility that produces the cell-processed product,
information on maintenance of the device in the facility that produces the cell-processed product,
environmental information on the facility that produces the cell-processed product, and
an environment maintenance method for the facility that produces the cell-processed product.

6. The information processing device according to claim 1, wherein
the predetermined quality standard is a preferable value or a range of the parameter in at least one type of the parameters included in the quality information.

7. The information processing device according to claim 1, wherein
the processor
generates, based on a plurality of ranges designated by the one or more types of the parameters, change recommendation information representing a recommended range for the one or more types of the parameters, and
the output device further outputs the change recommendation information.

8. The information processing device according to claim 1, wherein
the quality standard is a quality standard upon completion of the cell-processed product,
the processor determines pass or fail of the cell-processed product upon completion based on the first region and the quality information during production, and
the output device further outputs a pass or fail determination result.

9. The information processing device according to claim 1, wherein
the processor calculates, based on the quality information up to a first process, the quality state at a time when a second process after the first process ends, and
the first region is calculated based on a quality standard at a time when the second process ends.

10. A program for causing a computer to function as the information processing device according to claim 1.

11. An information processing method comprising:
a step of receiving, by an input device, quality information including a plurality of parameters related to a plurality of cell-processed products as an input, the quality information including a parameter related to at least one of production information, treatment information, treatment result information, and transport information related to the cell-processed products; a step of storing, by a storage device, a predetermined quality standard and the quality information;
a step of calculating, by a processor, a first region based on the quality standard;
a step of calculating, by the processor, a quality state of the cell-processed product based on the first region and the quality information;
a step of calculating, by the processor, a corresponding range of one or more other types of parameters based on a range designated by the one or more types of parameters; and
a step of outputting, by an output device, the corresponding range.
